(19) United States
(12) Patent Application Publication
(10) Pub. No.: US 2016/0058948 A1
(43) Pub. Date: Mar. 3, 2016

(54) DISPOSABLE SAFETY ANESTHETIC SYRINGE AND THE NEEDLE HUB THERETO

(71) Applicant: Horatio Yen, New Taipei City (TW)

(72) Inventor: Horatio Yen, New Taipei City (TW)

(21) Appl. No.: 14/471,784

(22) Filed: Aug. 28, 2014

(51) Int. Cl. A61M 5/24; A61M 5/34

(52) U.S. Cl. CPC A61M 5/2466; A61M 5/24; A61M 5/343; A61M 2005/3118

(57) ABSTRACT

The present invention discloses a disposable safety syringe that has a needle hub having an accommodating space therein for various purposes. More specifically, the present invention provides a needle hub, configured to an end of a barrel, wherein the needle hub comprises an internal needle, an external needle and a hub body. The hub body has an accommodating space formed therein, the internal needle is configured at a proximal end of the hub body, the external needle is configured at a distal end of the hub body, the internal needle and the external needle are connected to the accommodating space for various purposes. Furthermore, the present application further provides a multi-stage protector for avoiding needle prick accidents.

### FIELD OF THE INVENTION

The present invention discloses a disposable safety anesthetic syringe and the needle hub thereof, and more specifically, the present invention provides a needle hub having an accommodating space formed therein for various functions and a non-reusable disposable safety syringe having the said needle hub.

### BACKGROUND

Disposable syringes with retractable needles are desirable because they are economical and hygienic. Retractable needles are especially desirable because they greatly reduce accidental sticking of needles in caregivers and patients. Further, the retractable nature of the needles is advantageous because they provide a visual aid that can indicate prior use. In addition, many retractable designs make reusing the needle and syringe impossible.

Nowadays, the current designs of most re-usable syringe assemblies, the needle must be removed by hand. This greatly increases the risk of accidental being pricked by the needle. Therefore, the existing syringes usually offer a retractable needle and a break-away plunger to facilitate disposal and increase safety standards. More specifically, in order to avoid needle pricks, the existing syringes usually provides a needle retracting means for retracting a needle after use. Furthermore, some of the disposable syringe also utilizes a protecting cover to avoid accidental needle pricks, providing a means to solve the said problem.

In summary, it is clearly shown that most of the improvements of the syringe are focused on either lowering the cost by simplifying the structure or avoiding accidental needle pricks by having a retractable member or a protection cover

### SUMMARY OF THE INVENTION

However, the applicant has noticed that in some rare cases, especially in districts that have serious air pollution or poor environmental conditions, after the sealed cartridge that contains solution are withdrawn from aseptic packing, the surface of the cartridge may become polluted because of some unexpected reasons. In these situations, pollutants, such as dust, chemical or bacteria, may enter the solution casing and cause contamination.

In addition, avoiding contamination is not adequately addressed in the prior art, since syringes nowadays are used under the assumption that people use the syringes with a sterilized hand and in a clean environment. Accordingly, the applicant indicates that it is desirable to have a novel disposable syringe that incorporates the advantages of a contamination free syringe in order to minimize the risk of pollutants contaminating the syringe. Furthermore, for the cases that require the replacement of needle hub (for example, different length of needle required in various treatments), the needle hub of the present invention can also be replaceable.

More specifically, the present invention discloses a disposable safety syringe, for injecting a first solution in a sealed cartridge to a subject matter, which may comprises a barrel, a handle, a plunger and a needle hub. The barrel has a first opening and a corresponding second opening, the barrel extends along an extension direction; the handle is configured to the first opening of the barrel; the plunger has a rod, the rod penetrates through the handle so as to enter the barrel via the first opening thereof, for allowing the rod to slide along the extension direction inside the barrel; meanwhile, the needle hub is configured to the second opening of the barrel, the needle hub comprises an internal needle, an external needle and a hub body, the hub body has an accommodating space formed therein, the internal needle is configured at a proximal end of the hub body, the external needle is configured at a distal end of the hub body, the internal needle and the external needle are both connected to the accommodating space respectively; wherein, while the sealed cartridge is disposed in the barrel and the first solution in the sealed cartridge can be injected to the subject matter via the internal needle, the accommodating space and the external needle by applying a force to the plunger.

Accordingly, one of the aspects of the present invention is to provide a disposable safety anesthetic syringe that has the ability to filter out pollutants or other target subject matters in the injecting solution so as to improve the safety of syringe as present in the prior art.

The advantage and spirit of the invention may be understood by the following recitations together with the appended drawings.

### BRIEF DESCRIPTION OF THE DRAWINGS

FIG. 1 illustrates a schematic view of a preferred embodiment of the present invention.

FIG. 2 illustrates a schematic, cross-sectional view of the preferred embodiment of a needle hub of the present invention.

FIG. 3 illustrates a schematic, cross-sectional view of another embodiment of a needle hub of the present invention.

FIG. 4 illustrates a schematic, cross-sectional view of another embodiment of a needle hub of the present invention.

FIG. 5 illustrates a schematic, cross-sectional view of another embodiment of a needle hub of the present invention.

FIGS. 6A to 6D illustrate schematic views of a protector of the present invention in different phases.

FIG. 7 illustrates a schematic, cross-sectional view of another embodiment of a needle hub of the present invention.

### DETAILED DESCRIPTION

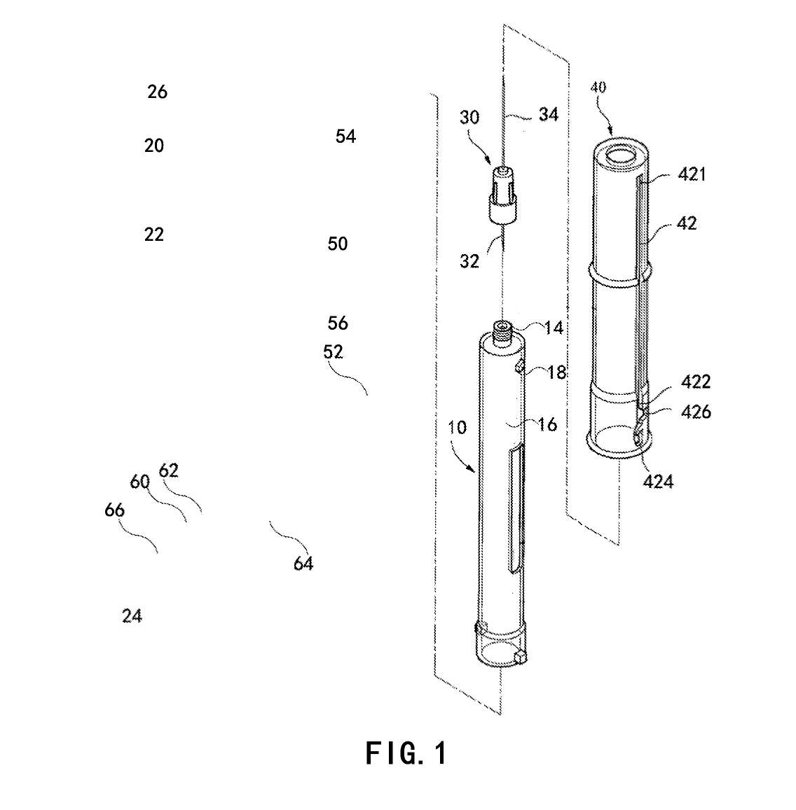

Please refer to FIG. 1. FIG. 1 illustrates a schematic sectional view of the disposable safety syringe of the present invention. More specifically, the present invention can be a syringe for anesthetic use. Please refer to FIG. 1. The disposable safety syringe 1 is mainly composed of a barrel 10, a handle 60, a plunger 20, a needle hub 30, a protector 40 and a sealed cartridge 50.

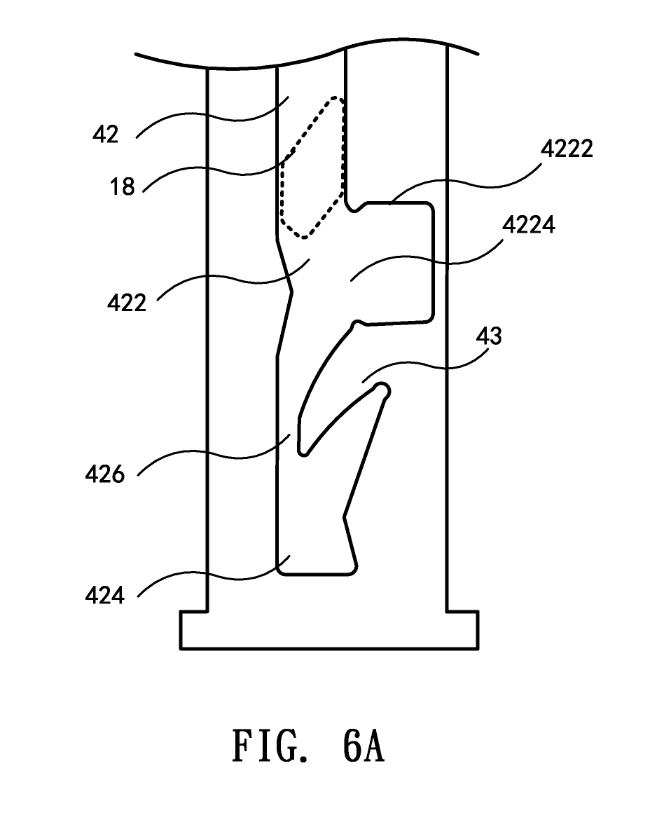

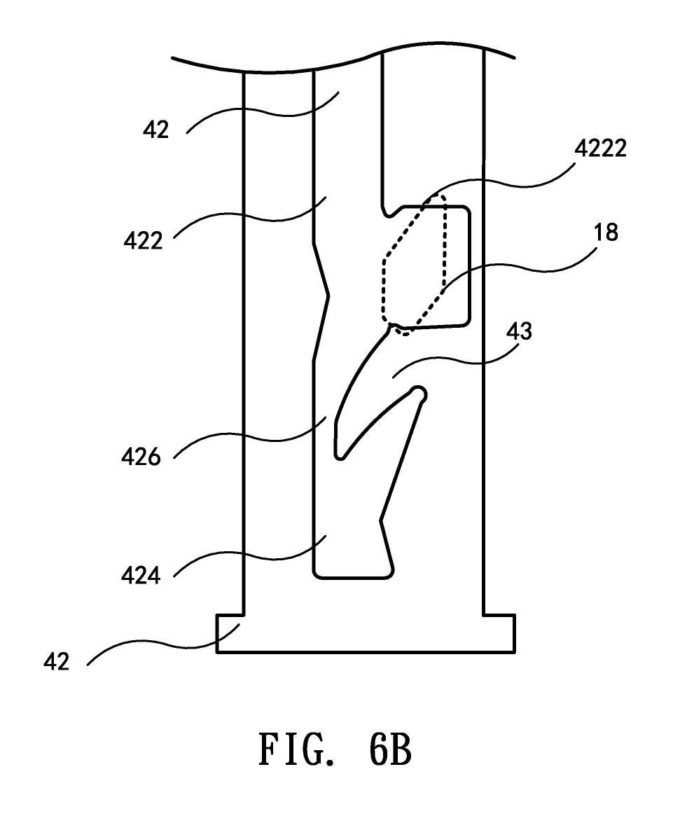

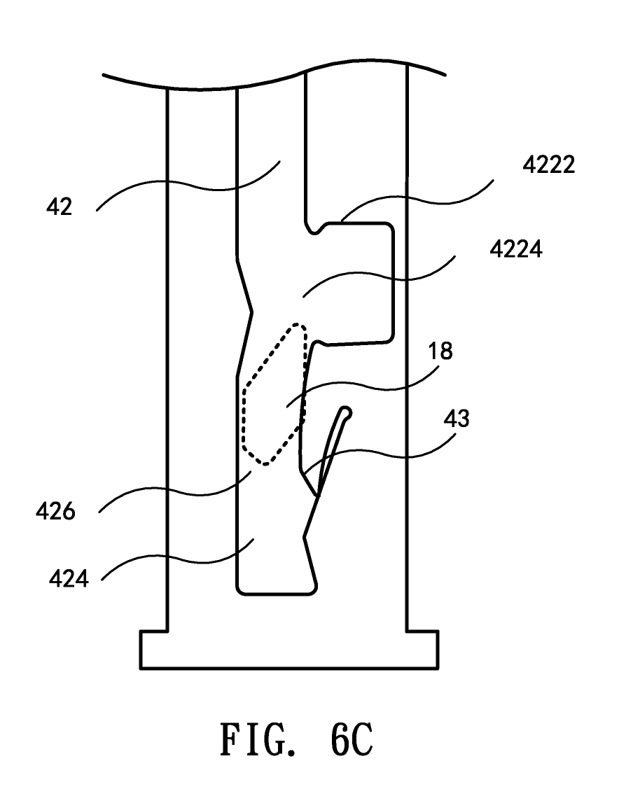

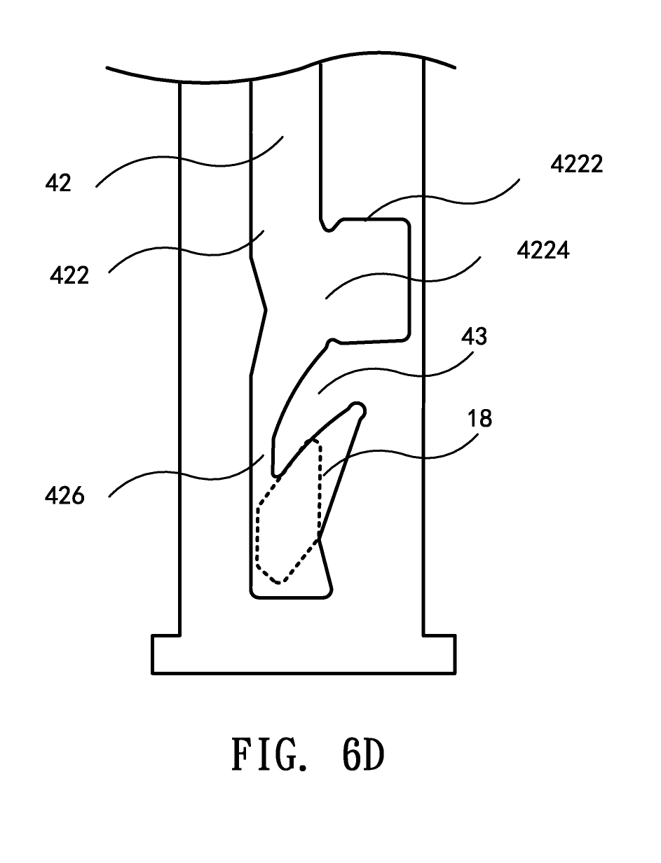

The barrel 10 has a first opening 12, a corresponding second opening 14 and a hollow space 16 connecting therebetween. The barrel 10 extends along an extension direction. The hollow space 16 allows the sealed cartridge 50 to be accommodated in the barrel 10. Furthermore, the linear surface of the barrel 10 has a protrusion 18 formed thereon, where the protrusion 18 has a sharp corner with an acute angle as depicted in FIG. 6A.

The sealed cartridge 50 is a rubber/glass material based solution having a sliding end 52 and an outputting end 54. The outputting end 54 is covered with a soft material, for example rubber, allowing it to be penetrated by a needle so as to output the containing solution therefrom. In the present embodiment, the solution is a liquid based anesthetic solution. The sliding end 52 has a plug 56; the plug 56 can be pressed into the sealed cartridge 50 to output the solution in the cartridge 50.

The handle 60 has a main body 62 with a ring 64 and an ear 66. The ring 64 and the ear 66 are connected on the corresponding linear surface of the handle 60 and extend outward therefrom for a user to hold. The plunger 20 comprises a rod 22 and a pushing ring 24, where the rod 22 penetrates through the main body 62 of the handle 60 so as to enter the barrel 10 via the first opening 12 thereof, allowing the rod 22 to slide along the extension direction. One of the ends of the rod 22 is configured with the pushing ring 24, allowing the thumb of the user to apply a force to the rod 22 for the said sliding motion. Furthermore, another end of the rod 22 has a contact portion 26, while the diameter of the contact portion 26 is slightly smaller than the diameter of the plug 56, allowing the rod 22 to push the plug 56 forward in order to output the solution. Furthermore, in the present embodiment, the contact portion 26 comprises a drill member for fixing with the rod 22 with the plug 56 so as to allow the user to inhale solution to accommodating space 362 from outside space via the needle hub 30 by pulling the rod 22 backward.

It should be noticed that the whole plunger 20 and handle 60, including the ring 64 and the ear 66 can be formed from a polymer based material for low cost and acceptable strength.

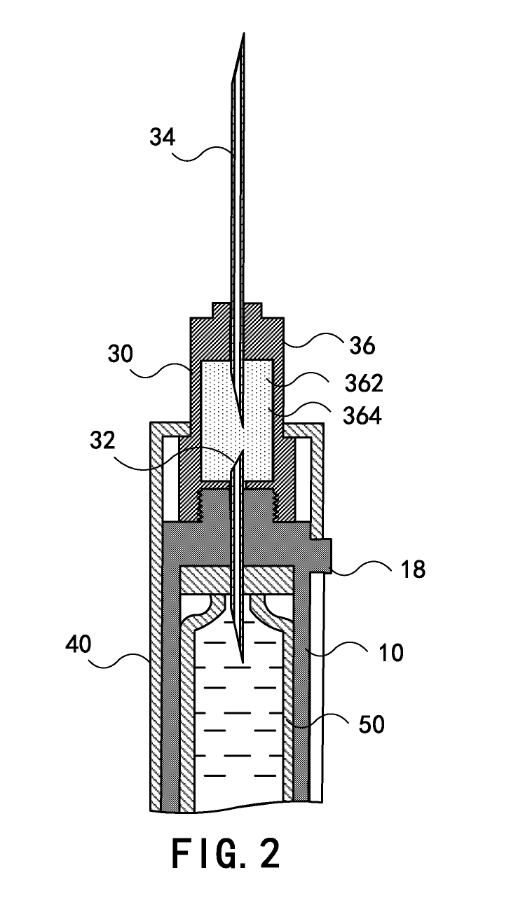

Please refer to FIG. 2, which depicts a preferred embodiment of a needle hub 30 of the present invention. The needle hub 30 comprises a hub body 36, an internal needle 32 and an external needle 34. The hub body 36 has a fixing portion 364 formed on the linear surface of the proximal end thereof. The fixing portion 364 can be fixed with the barrel 10 by the screw threads thereon. Furthermore, the hub body 36 further has an accommodating space 362 formed therein, allowing a predetermined matter 364 corresponding to a certain purposes to be accommodated therein. For example, the said matter may be a filtering material providing a filtering function which has the ability to filter out predetermined pollutants in the solution. For example, dust, chemical or even bacteria. The internal needle 32 and the external needle 34 penetrate through the proximal end and the distal end of the hub body 36 respectively and are fixed thereat. The internal needle 32 and the external needle 34 are connected to the accommodating space 362. In actual practice, the internal needle 32 enters the barrel 10 via the second opening 14 formed at the distal end of the barrel 10.

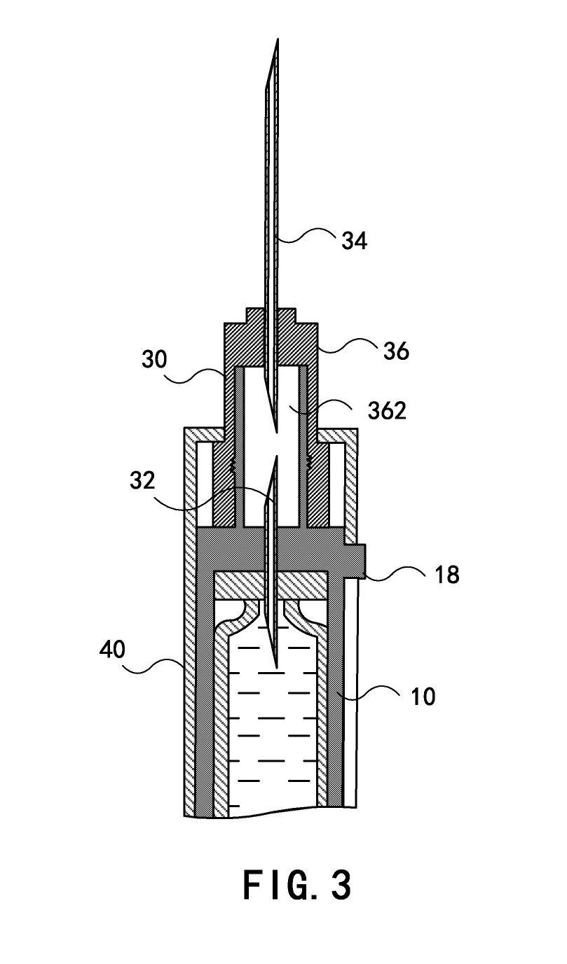

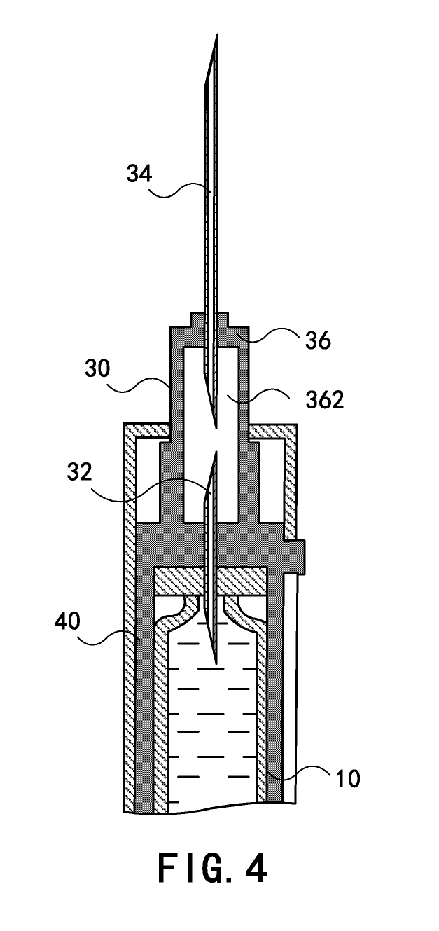

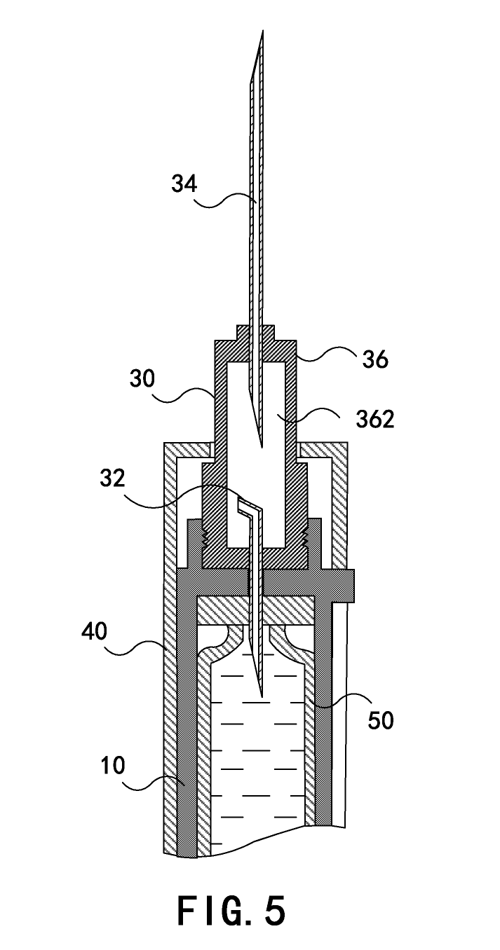

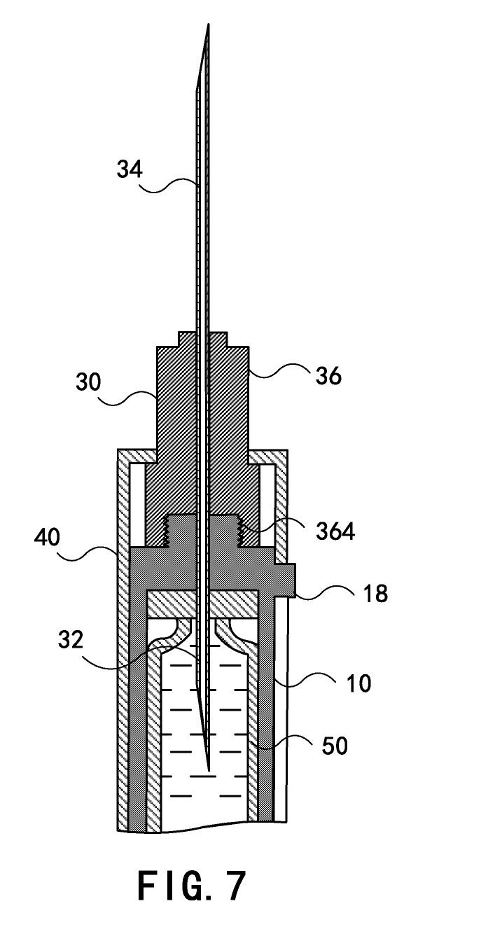

In another embodiment depicted as FIG. 3, the needle hub 30 is not necessarily formed as one piece. More specifically, the accommodating space 362 can be formed by the hub body 36 and the barrel 10. In this embodiment, the internal needle 32 is now fixed in the second opening 14 as a part of the needle hub 30. Please refer to FIG. 4. In the present embodiment, as depicted, apart from the needles, the whole hub body 36 of the needle hub 30 can even be one piece formed with the barrel 10 for better strength. Moreover, in the embodiment of FIG. 3, the internal needle 32 is aligned with the external needle 34 in the accommodating space 362. However, the internal needle 32 can also have an offset to the external needle 34 as depicted in FIG. 5. More specifically, the internal needle 32 may be twisted for certain purposes, such as increasing the turbulence for a better mixing effect. Furthermore, in another embodiment, the internal needle 32 and the external needle 34 may be connected and formed as one piece and the accommodating space may be omitted therefrom as depicted in FIG. 7.

Please refer to FIG. 1 and FIG. 6A to 6D. In the present embodiment, the invention further comprises a protector 40. The hollow protector 40 has a groove 42 and a hook 43 formed on the linear surface 421 of the groove 42, where the hook 43 divides the groove 42 (or called as guiding groove 42) into a sliding portion 422, a fixing portion 424 and a tunnel portion 426 disposed therebetween. The sliding portion 422 of the groove 42 has a concave 4222 formed at the end of the sliding portion 422 corresponding to the first opening 12, where the concave 4222 allows the protrusion 18 to be disposed and fixed therein. Furthermore, the concave 4222 has an entrance 4224. The entrance 4224 is in a tight fit with the protrusion 18 to allow the protrusion 18 to enter or exit the concave 4222 under a certain amount of pressure. By the condition of the protector 40, the disposable safety syringe 1 may be classified in various phases, which are the pre-use phase, used phase and holding phase.

Please refer to FIG. 6A. The FIG. 6A depicts a pre-use phase of the protector. As depicted, a protrusion 18 is formed on the surface of the barrel 10 and is moveably configured in the sliding portion 422 of the groove 42.

Please refer to FIG. 6B, the FIG. 6B depicts a holding phase of the protector. In the holding phase, the protrusion 18 is in the concave 4222 and the whole needle hub 30 is in the protector, thus protecting the user from being pricked by the needle accidentally and also reserving the ability of re-using of the syringe 1.

After being used, in order to protect the user from accentually being pricked by the needle, the user may switch the disposable safety anesthetic syringe 1 switch from the pre-use phase to the used phase by pulling the protector 40 rearward in order to move the protrusion 18 from the sliding portion 422 to the fixing portion 424 via the tunnel portion 426 as depicted in FIG. 6C. While the protrusion 18 is passing through the tunnel portion 426, the protrusion 18 presses the hook 43 and the hook 43 plastically deforms accordingly so as to enlarge the tunnel portion 426 to allow the protrusion 18 to pass therethrough. After the protrusion 18 passes through the tunnel portion 426, the pressure applied thereto is gone, and the hook 43 recovers to the initial shape thereof and the sharp corner of the protrusion will be embedded in the hook 43 and fixed therewith in order to prevent the protrusion 18 from re-entering the sliding portion. Meanwhile, the whole external needle 34 is contained in the protector 40 to avoid the re-usage thereof.

In summary, while the disposable safety anesthetic syringe 1 is in the pre-use phase, the protrusion 18 formed on the surface of the barrel 10 is moveably configured in the sliding portion of the groove 42. However, in the used phase, the protrusion 18 formed on the surface of the barrel 10 is disposed and fixed in the fixing portion. Meanwhile, the whole needle hub 30 is disposed in the hollow protector 40 in order to avoid possible accidents.

Please refer to the FIG. 1. While in actual practice, the needle hub 30 is first configured to the second opening 14 of the barrel 10. The sealed cartridge 50 is then disposed into the hollow space of the barrel 10, and then the internal needle 32 of the needle hub 30 penetrates through the soft end of the sealed cartridge 50. The handle 60 with the plunger 20 that penetrates therethrough is then configured and fixed to the first opening 12 of the barrel 10. Then, the user may hold the handle 60 and push the pushing ring 24 of the plunger 20 forward so as to drive the rod 22 toward the second opening 14. Accordingly, the rod 22 presses the plug 56 into the sealed cartridge 50 in order to compress the solution to output the solution from the internal needle 32 to the external needle 34 via the accommodating space 362, after the treatment done to the solution in the accommodating space 362. Furthermore, the handle can be formed as one pieced with the barrel.

In summary, the present invention discloses a disposable safety syringe that has a needle hub that has an accommodating space therein for various purposes. Furthermore, the present application further provides a multiple stage protector to avoid accidental needle pricks.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

1. A disposable safety syringe, for injecting a first solution in a sealed cartridge to a subject matter, comprising:
a barrel, having a first opening and a corresponding second opening, the barrel extending along an extension direction;
a handle, configured to the first opening of the barrel;
a plunger, having a rod, the rod penetrating through the handle so as to enter the barrel via the first opening thereof, for allowing the rod to slide along the extension direction inside the barrel; and
a needle hub, configured to the second opening of the barrel, the needle hub comprising an internal needle, an external needle and a hub body, the hub body having an accommodating space formed therein, the internal needle configured at a proximal end of the hub body, the external needle configured at a distal end of the hub body, the internal needle and the external needle connected to the accommodating space respectively;
wherein, while the sealed cartridge is disposed in the barrel and the first solution in the sealed cartridge can be injected to the subject matter via the internal needle, the accommodating space and the external needle by applying a force to the plunger.

2. The disposable safety syringe of claim 1, wherein the barrel, the handle and the plunger are formed of high polymer based material.

3. The disposable safety syringe of claim 1, further comprising a hollow protector, the hollow protector having a groove and a hook formed on the linear surface of the groove, the hook dividing the groove into a sliding portion, a fixing portion and a tunnel portion disposed therebetween; wherein the disposable safety syringe has a pre-use phase and a used phase, in the pre-use phase, a protrusion formed on the linear surface corresponding to the second opening of the barrel is moveably configured in the sliding portion of the groove, in the used phase, the protrusion is fixed with the hook in the fixing portion, meanwhile, the whole needle hub is disposed in the hollow protector.

4. The disposable safety syringe of claim 3, wherein, the hook extends rearward from the latent surface of the groove, while the disposable safety syringe is switched from the pre-use phase to the used phase, the protrusion slides from the sliding portion to the fixing portion via the tunnel portion, while the protrusion passes the tunnel portion, the protrusion presses and plastically deforms the hook so as to enlarge the tunnel portion for allowing the protrusion to pass therethrough.

5. The disposable safety syringe of claim 3, wherein the sliding portion of the groove has a concave formed at the end of the sliding portion corresponding to the first opening, the concave allows the protrusion to be disposed therein for fixation.

6. The disposable safety syringe of claim 5, wherein the concave has an entrance and the entrance is in tight fit with the protrusion.

7. The disposable safety syringe of claim 3, wherein, the protrusion has a sharp corner with an acute angle, while the disposable safety syringe is in the used phase, the sharp corner is embedded in the hook and fixed therewith so as to prevent the protrusion from re-entering the tunnel portion.

8. The disposable safety syringe of claim 1, wherein the internal needle is staggered to the external needle.

9. The disposable safety syringe of claim 1, wherein the accommodating space has a filtering member disposed therein for filtering the first solution.

10. The disposable safety syringe of claim 1, wherein the first solution is an anesthetic.

11. The disposable safety syringe of claim 1, wherein the needle hub is formed as one pieced with the barrel.

12. The disposable safety syringe of claim 1, wherein the handle is formed as one pieced with the barrel.

13. A needle hub for a syringe, comprising an internal needle, an external needle and a hub body, the hub body having an accommodating space formed therein, the internal needle configured at a proximal end of the hub body, the external needle configured at a distal end of the hub body, the internal needle and the external needle connected to the accommodating space respectively;
wherein, while the needle hub is connected with a barrel with a sealed cartridge disposed therein and a first solution in the sealed cartridge can be injected to a subject matter via the internal needle, the accommodating space and the external needle.
